(19) United States
(12) Plant Patent Application Publication
(10) Pub. No.: US 2026/0114345 P1
(43) Pub. Date: Apr. 23, 2026

(54) Veronica Plant Named 'Balskywac'

(50) ;

(71) Applicant: Ball Horticultural Company, West Chicago, IL (US)

(72) Inventors: Jessica Diane Janes, Elkton, MD (US); Laura M. Lara, Traverse City, MI (US)

(21) Appl. No.: 19/359,059

(22) Filed: Oct. 15, 2025

Related U.S. Application Data
(60) Provisional application No. 63/709,360, filed on Oct. 18, 2024.

(51) Int. Cl. A01H 6/68; A01H 5/02; A01H 5/12

(52) U.S. Cl. CPC A01H 6/68; A01H 5/02; A01H 5/12
USPC PLT/251

(57) ABSTRACT

A new and distinct cultivar of Veronica plant named ‘Balskywac’, characterized by its light, purple-colored flowers, medium, green-colored foliage, and moderately vigorous, compact, upright-mounded growth habit, is disclosed.

### CROSS REFERENCE TO RELATED APPLICATIONS

This application claims the benefit of U.S. Provisional Application No. 63/709,360 filed Oct. 18, 2024.

Latin name of genus and species of plant claimed:

Veronica hybrid

Variety denomination:

‘Balskywac’.

### BACKGROUND OF THE INVENTION

The present invention relates to a new and distinct cultivar of Veronica plant botanically known as Veronica hybrid and hereinafter referred to by the cultivar name ‘Balskywac’.

The new cultivar originated in a controlled breeding program in West Grove, Pennsylvania, during May 2019. The objective of the breeding program was the development of Veronica cultivars that flower without a vernalization requirement, have attractive and numerous inflorescences, and a compact, upright-mounded growth habit.

The new Veronica cultivar was the result of cross-pollination. The female (seed) parent of the new cultivar is Veronica ‘Royal Rembrandt’, U.S. Plant Pat. No. 29,377, characterized by its medium, purple-colored flowers, dark, green-colored foliage, and moderately vigorous, mounded growth habit. The male (pollen) parent of the new cultivar is proprietary Veronica spicata breeding selection X15-6303-002, not patented, characterized by its medium blue-violet-colored flowers, medium, green-colored foliage, and moderately vigorous, compact, upright-mounded growth habit. The new cultivar was discovered and selected as a single flowering plant within the progeny of the above stated cross-pollination during July 2021 in a controlled environment in West Grove, Pennsylvania.

Asexual reproduction of the new cultivar by terminal stem cuttings since July 2021 in West Grove, Pennsylvania and Elburn, Illinois has demonstrated that the new cultivar reproduces true to type with all the characteristics, as herein described, firmly fixed and retained through successive generations of such asexual propagation.

### SUMMARY OF THE INVENTION

The following characteristics of the new cultivar have been repeatedly observed and can be used to distinguish ‘Balskywac’ as a new and distinct cultivar of Veronicaplant:
1. Light, purple-colored flowers.
2. Medium, green-colored foliage; and
3. Moderately vigorous, compact, upright-mounded growth habit.

Plants of the new cultivar differ from plants of the female parent primarily in having more light purple coloration in the flower color, lighter, green-colored foliage, and an upright growth habit. Plants of the new cultivar differ from plants of the male parent primarily in having a purple flower color.

Of the many commercially available Veronica cultivars, the most similar in comparison to the new cultivar is Veronica ‘Purpleicious’, U.S. Plant Pat. No. 17,639. However, in side-by-side comparisons, plants of the new cultivar differ from plants of ‘Purpleicious’ in at least the following characteristics:
1. Plants of the new cultivar has a more compact growth habit than plants of ‘Purpleicious’;
2. Plants of the new cultivar have shorter leaves than plants of ‘Purpleicious’.

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs may differ slightly from the color values cited in the detailed description, which accurately describes the colors of ‘Balskywac’. The plants were approximately four months old and grown in 4-inch pots for approximately 12 weeks in a greenhouse in West Chicago, Illinois. Plants were given one pinch at approximately three weeks after transplant.

FIG. 1 illustrates a side view of the overall growth and flowering habit of ‘Balskywac’.

FIG. 2 illustrates a close-up view of the inflorescences of ‘Balskywac’.

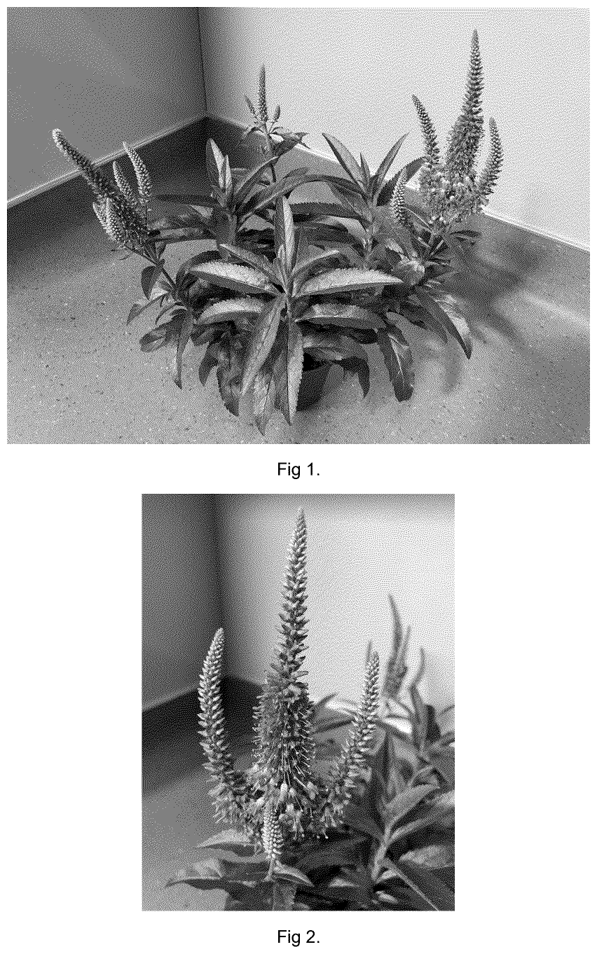

### DETAILED BOTANICAL DESCRIPTION

The new cultivar has not been observed under all possible environmental conditions to date. Accordingly, it is possible that the phenotype may vary somewhat with variations in the environment, such as temperature, light intensity, and day length, without, however, any variance in genotype.

The chart used in the identification of colors described herein is The R.H.S. Colour Chart of The Royal Horticultural Society, London, England, 2015 edition, except where general color terms of ordinary significance are used. The color values were determined in March 2025 under natural light conditions in West Chicago, Illinois. The following descriptions and measurements describe approximately four-month-old plants produced from cuttings from stock plants and grown under conditions comparable to those used in commercial practice. The plants were grown for approximately 12 weeks in a greenhouse in West Chicago, Illinois. Plants were given one pinch at approximately three weeks after transplant. Greenhouse temperatures were maintained at approximately 60° F. to 65° F. (15.6° C. to 18.3° C.) during the day and approximately 55° F. to 60° F. (12.8°° C. to 15.6° C.) during the night. Supplemental lighting was used. Measurements and numerical values represent averages of typical plants.
Botanical classification: Veronica spicata ‘Balskywac’.
Parentage:
   Female parent.—Veronica ‘Royal Rembrandt’, U.S. Plant Pat. No. 29,377.
   Male parent.—Proprietary Veronica spicata breeding selection X15-6303-002, not patented.
Propagation:
   Type cutting.—Terminal stem.
   Time to initiate roots.—Approximately 7 to 10 days.
   Time to produce a rooted cutting.—Approximately 35 to 42 days.
   Root description.—Fibrous.
   Rooting habit.—Freely branching.
Plant description:
   Commercial crop time.—Approximately 10 to 12 weeks from a rooted cutting to finish in a 15 cm pot. Vernalization to initiate flowering is not required.
   Growth habit and general appearance.—Herbaceous perennial, moderately vigorous, compact, upright-mounded.
   Hardiness.—USDA Zone 4b (−25° F. to −20° F./−31.7° C. to −28.9° C.).
   Size.—Height from soil level to top of plant plane: Approximately 29.0 cm. Width: Approximately 32.0 cm.
   Branching habit.—Freely branching, pinching enhances basal branching. Quantity of main branches per plant: Approximately 8.
   Branch.—Strength: Moderate. Branch height to bottom of inflorescence: Approximately 23.0 cm. Diameter: Approximately 3.0 mm. Length of central internode: Approximately 3.0 cm. Texture: Moderately pubescent. Color of young stems: 144A. Color of mature stems: 144B, becoming woody with an overlay of N199C with age.
Foliage description:
   General description.—Quantity of leaves per main branch: Approximately 10. Fragrance: None detected. Form: Simple. Arrangement: Opposite, decussate.
   Leaves.—Aspect: Perpendicular to stem. Shape: Lanceolate. Margin: Serrate. Apex: Acute to broadly acute. Base: Attenuate. Venation pattern: Pinnate. Length of mature leaf: Approximately 8.0 cm. Width of mature leaf: Approximately 2.4 cm. Texture of upper surface: Sparsely pubescent. Texture of lower surface: Sparsely pubescent and moderately pubescent on venation. Color of upper surface of young and mature foliage: Closest to NN137A. Color of lower surface of young and mature foliage: Closest to 137B. Venation of upper and lower surface: 146B.
   Petioles.—Length: Approximately 8.0 mm. Width: Approximately 4.0 mm. Texture: Sparsely pubescent. Color: 146C.
Flowering description:
   Flowering habit.—‘Balskywac’ is a long-day obligate, freely flowering under outdoor growing conditions blooming from early spring through autumn.
   Lastingness of individual flower on the plant.—Approximately 7 days.
Inflorescence description:
   General description.—Type: Terminal raceme, self-cleaning. Fragrance: None detected. Height: Approximately 9.0 cm to 13.0 cm. Width: Approximately 1.5 cm to 2.0 cm. Quantity per plant: Approximately 20.
   Peduncle.—Strength: Strong. Aspect: Erect. Length: Approximately 2.5 cm to 5.0 cm. Diameter: Approximately 1.5 mm to 3.0 mm. Texture: Densely pubescent. Color: 144A.
Flower description:
   General description.—Type: Single, salverform. Aspect: Outward. Quantity per inflorescence: Approximately 90.
   Bud.—Rate of opening: Generally, takes 3 to 4 days for bud to progress from first color to fully open flower.
   Bud just before opening.—Shape: Oblong. Length: Approximately 6.0 mm. Diameter: Approximately 2.0 mm. Texture: Glabrous. Color: 77A with NN 155A at base.
   Corolla.—Height: Approximately 7.0 mm. Diameter: Approximately 8.0 mm.
   Petals.—Quantity: 4. Appearance: Dull. Shape: Obelliptic. Margin: Entire, wavy. Apex: Broadly acute. Base: Fused into tube. Length: Approximately 5.0 mm. Width: Approximately 2.0 mm to 5.0 mm. Texture of upper and lower surfaces: Glabrous. Color of upper and lower surfaces when first open: N81A. Color of upper and lower surfaces when fully open N81A to N81B.
   Corolla tube.—Length: Approximately 2.0 mm. Diameter at tube opening: Approximately 2.0 mm. Texture of inner surface: Densely pubescent. Color of pubescence: NN155D. Texture of outer surface: Glabrous. Color of inner surface: NN155D. Color of outer surface: NN155D with a faint overlay of N81B.
   Calyx.—Shape: Cupped. Length: Approximately 3.0 mm. Diameter: Approximately 3.0 mm.
   Sepals.—Quantity per flower: 4. Shape: Lanceolate, fused at base. Apex: Acute. Length: Two larger sepals approximately 3.0 mm and two smaller sepals approximately 2.0 mm. Width of all sepals: Approximately 1.0 mm. Texture of upper (inner) surface: Glabrous. Texture of lower (outer) surface: Moderately pubescent and densely pubescent on margins. Color of upper and lower surfaces: 137A with 146D at base.
   Pedicel.—Strength: Strong, flexible. Aspect: Approximately 45° angle from peduncle. Length: Approximately 1.0 mm. Diameter: Approximately 0.5 mm. Texture: Densely pubescent. Color: 146B.
   Reproductive organs.—Androecium: Stamen quantity: 2 per flower. Stamen length: Approximately 7.0 mm. Filament color: 76A. Anther shape: Sagittate, dorsifixed. Anther length: Approximately 1.0 mm. Anther color: 79A. Pollen amount: Abundant. Pollen color: 2D. Gynoecium: Pistil quantity: 1 per flower. Pistil length: Approximately 1.0 cm. Stigma shape: Rounded. Stigma length: Less than 1.0 mm. Stigma color: 79A. Style length: Approximately 7.0 mm. Style color: 79D. Ovary diameter: Approximately 1.0 mm. Ovary color: 145A.
Seed and fruit production: Neither seed nor fruit production has been observed.
Disease and pest resistance: Resistance to pathogens and pests common to Veronica has not been observed.

What is claimed is:

1. A new and distinct cultivar of Veronica plant named ‘Balskywac’, substantially as herein illustrated and described.
